(19) United States
(12) Patent Application Publication
(10) Pub. No.: US 2025/0155553 A1
(43) Pub. Date: May 15, 2025

(54) SYSTEM AND METHOD FOR THE CONTACTLESS RECORDING OF VITAL PARAMETERS BY CREATING A BODY MODEL BASED ON BODY SUBDIVISION ALONG THE RADAR FIELD OF VIEW

(71) Applicant: Fraunhofer-Gesellschaft zur Förderung der angewandten Forschung e.V., München (DE)

(72) Inventors: Lars HORNIG, Oldenburg (DE); Jens-Ekkehart APPELL, Oldenburg (DE)

(21) Appl. No.: 19/024,543

(22) Filed: Jan. 16, 2025

Related U.S. Application Data
(63) Continuation of application No. PCT/EP2023/073065, filed on Aug. 22, 2023.

(30) Foreign Application Priority Data
Aug. 29, 2022 (DE) 10 2022 208 945.6

(51) Int. Cl. G01S 7/41; A61B 5/05; G01S 13/88

(52) U.S. Cl. CPC G01S 7/41; A61B 5/05; G01S 13/88

(57) ABSTRACT

A system for determining information about one or more vital parameters of a human being includes a radar device for emitting first radar waves and detecting reflected radar waves caused by reflection of the first radar waves at the human being or at a body cover of the human being. The system includes an evaluation unit for determining information about the vital parameters depending on the reflected radar waves. The radar device is arranged, relative to the position of the human being, laterally with respect to the human being such that there is a virtual point in a foot of the human being and such that there is a virtual point in the radar device so that the virtual point in the radar device is closer to the virtual point in the foot of the human being than to any virtual point in a navel of the human being.

### CROSS-REFERENCES TO RELATED APPLICATIONS

This application is a continuation of copending International Application No. PCT/EP2023/073065, filed Aug. 22, 2023, which is incorporated herein by reference in its entirety, and additionally claims priority from German Application No. DE 10 2022 208 945.6, filed Aug. 29, 2022, which is incorporated herein by reference in its entirety.

The application concerns the contactless recording of vital parameters and in particular a system and a method for the contactless recording of vital parameters by creating a body model based on body subdivision along a radar field of view.

### BACKGROUND OF THE INVENTION

In particular, the present invention concerns monitoring of vital parameters. As people are not aware of their condition during sleep, monitoring vital parameters during sleep is often beneficial. For example, this applies to people with illnesses such as chronic respiratory disorders. Ideally, to this end, people should not be disturbed during sleep. However, measuring devices for vital parameters are usually contact-based, which is why such devices are usually attached to the sleeping person. As a result, the measuring devices are expensive to install and maintain and are less comfortable for the patient. In contrast, it is beneficial to be able to measure as many vital parameters as a possible in a robust, valid, and contactless way with few measuring devices.

Contactless measuring devices based on distance measurements between a person and a sensor can be used for breathing, heartbeat, and movements of the body and limbs.

The movement of the person leads to a characteristic change in distance that can be assigned to a vital parameter. For example, the abdominal region will inflate when breathing, and the abdominal surface will move closer to the measuring device. When exhaling, the abdomen returns to its initial state, and the abdominal surface moves away from the measuring device. This should occur at a frequency that is characteristic of breathing (e.g. 10-24 times per minute). The same occurs for different vital parameters simultaneously over the entire body (heartbeat at the upper chest, kicking motion at the legs).

In this case, the problem is the simultaneous superimposition of many vital parameters in the measuring data. Breathing, heartbeat, and limb movements often occur simultaneously and lead to a complex signal that then has to be broken down into individual vital parameters. When analyzing the data of the contactless measuring device, it is not always clear which movements form the basis of this complex signal. As a result, contactless determination of vital parameters is not trivial, which makes the use of contactless measuring devices more difficult.

For contactless determination of vital parameters by means of distance measurements, radar or ultra-sound devices have been previously used to emit and capture (or record) the signal.

US 2022/0142478 A1 and WO 2021/086809 A1 show the use of a radar to particularly distinguish only between heartbeat and breathing.

Until now, the radar has been positioned in front of or behind the upper body, i.e. above or below the bed when a person is in bed. Some cases use only radar positions in front of or behind the body. These positions mean that either only a section of the body is observed or the entire body is at a similar distance from the measuring device. In these positions, the heartbeat and breathing are most clearly recognizable for the radar, as the body surface moves directly toward the radar during the movements. However, in this case, they also overlap to a maximum, as breathing and heartbeat movements occur in the same direction.

The current standard solution for the superimpositions is the use of complex algorithms to separate the individual signals from each other.

However, due to the complexity of the evaluation, inappropriate values in the vital parameters are not easily comprehended. In addition, the complexity of the underlying problem also leads to breaks in which the vital parameters cannot be extracted from the signal. In addition, the database will be a superimposition of different movements. As a result, the validity of the results can only ever be ensured at theoretical limits for, e.g., maximum/minimum sensible respiratory rate or heartrate. However, as a result, validation on the person, e.g. by assigning the movements to body parts, is not possible.

It would be desirable to provide improved concepts for the contactless recording of vital parameter.

### SUMMARY

An embodiment may have a system for determining information about one or more vital parameters of a human being, the system comprising: a radar device for emitting first radar waves and detecting reflected radar waves caused by reflection of the first radar waves at the human being or at a body cover of the human being, and an evaluation unit for determining information about the one or more vital parameters of the human being depending on the reflected radar waves, wherein the radar device is arranged, relative to the position of the human being, laterally with respect to the human being such that there is a virtual point in a foot of the human being and such that there is a virtual point in the radar device so that the virtual point in the radar device is closer to the virtual point in the foot of the human being than to any virtual point in a navel of the human being; or wherein the radar device is arranged, relative to the position of the human being, laterally with respect the human being such that there is a virtual point in a head of the human being and such that there is a virtual point in the radar device so that the virtual point in the radar device is closer to the virtual point in the head of the human being than to any virtual point in the navel of the human being.

Another embodiment may have a method for determining information about one or more vital parameters of a human being, the method comprising: emitting first radar waves by a radar device, detecting, by the radar device, reflected radar waves caused by reflection of the first radar waves at the human being or at a body cover of the human being, and determining, by an evaluation unit, the information of the one or more vital parameters of the human being depending on the reflected radar wave, and wherein the radar device is arranged, relative to the position of the human being, laterally with respect to the human being such that there is a virtual point in a foot of the human being and such that there is a virtual point in the radar device so that the virtual point in the radar device is closer to the virtual point in the foot of the human being than to any virtual point in a navel of the human being; or wherein the radar device is arranged, relative to the position of the human being, laterally with respect the human being such that there is a virtual point in a head of the human being and such that there is a virtual point in the radar device so that the virtual point in the radar device is closer to the virtual point in the head of the human being than to any virtual point in the navel of the human being.

Another embodiment may have a non-transitory digital storage medium having a computer program stored thereon to perform the method for determining information about one or more vital parameters of a human being, the method comprising: emitting first radar waves by a radar device, detecting, by the radar device, reflected radar waves caused by reflection of the first radar waves at the human being or at a body cover of the human being, and determining, by an evaluation unit, the information of the one or more vital parameters of the human being depending on the reflected radar wave, and wherein the radar device is arranged, relative to the position of the human being, laterally with respect to the human being such that there is a virtual point in a foot of the human being and such that there is a virtual point in the radar device so that the virtual point in the radar device is closer to the virtual point in the foot of the human being than to any virtual point in a navel of the human being; or wherein the radar device is arranged, relative to the position of the human being, laterally with respect the human being such that there is a virtual point in a head of the human being and such that there is a virtual point in the radar device so that the virtual point in the radar device is closer to the virtual point in the head of the human being than to any virtual point in the navel of the human being, when said computer program is run by a computer.

A system for determining information about one or more vital parameters of a human being is provided according to an embodiment. The system includes a radar device for emitting first radar waves and detecting reflected radar waves caused by reflection of the first radar waves at the human being or at a body cover of the human being. In addition, the system includes an evaluation unit for determining information about the one or more vital parameters of the human being depending on the reflected radar waves. The radar device is arranged, relative to the position of the human being, laterally with respect to the human being such that there is a virtual point in a foot of the human being and such that there is a virtual point in the radar device so that the virtual point in the radar device is closer to the virtual point in the foot of the human being than to any virtual point in a navel of the human being. Or the radar device is arranged, relative to the position of the human being, laterally with respect the human being such that there is a virtual point in a head of the human being and such that there is a virtual point in the radar device so that the virtual point in the radar device is closer to the virtual point in the head of the human being than to any virtual point in the navel of the human being.

According to an embodiment, e.g., the radar device may be arranged relative to the position of the human being such that, instead of behind the human being, the radar device is positioned in front of the human being.

In an embodiment, e.g., the radar device may be arranged, relative to the position of the human being, laterally with respect to the human being such that the radar device is arranged below the navel of the human being and below the foot of the human being, or such that the radar device is arranged above the navel of the human being and above the head of the human being.

According to an embodiment, e.g., the radar device may be configured to detect one or more detected radar signals depending on the reflected radar waves, wherein each detected radar signal of the one or more detected radar signals is assigned to precisely one distance step of two or more distance steps. In this case, e.g., each of the two or more distance steps may be assigned to body region of a plurality of body regions of the human being so that the detected radar signal, assigned to this distance step, of the one or more detected radar signals is assigned to said body region. In this case, e.g., the evaluation unit may be configured to determine information about one of the one or more vital parameters depending on which detected radar signal of the one or more detected radar signals is assigned to which body region of the plurality of body regions.

In an embodiment, each of the two or more distance steps may be assigned to a body region, e.g. of a plurality of body regions of the human being, so that a body of the human being is fully subdivided into the plurality of body regions by the two or more distance steps so that the plurality of body regions together cover the body of the human being.

According to an embodiment, e.g., the one or more detected radar signals may be two or more detected radar signals. In this case, e.g., the radar device may be configured to detect the two or more detected radar signals, wherein each detected radar signals of the two or more detected radar signals is assigned to precisely one distance step of the two or more distance steps. For example, each of the two or more distance steps may be assigned to a body region of the plurality of body regions of the human being so that the detected radar signal, assigned to this distance step, of the two or more detected radar signals is assigned to said body region. In this case, e.g., the evaluation unit may be configured to determine information about said one of the one or more vital parameters depending on which detected radar signal of the two or more detected radar signals is assigned to which body part of the plurality of body parts.

In an embodiment, e.g., depending on the two or more detected radar signals, the evaluation unit may be configured to create a body model of the human being, indicating which radar signal is assigned to which body region of the plurality of body regions of the human being.

In an embodiment, e.g., each of the two or more detected radar signals may be assigned to precisely one body region, having assigned no other of the two or one more detected radar signals, of the two or more body regions.

According to an embodiment, e.g., the one or more vital parameters may be two or more vital parameters. In this case, e.g., the evaluation unit may be configured to determine information about the two or more vital parameters depending on which detected radar signal of the two or more detected radar signals is assigned to which body region of the plurality of body regions.

In an embodiment, e.g., the two or more detected radar signals may be three or more detected radar signals, wherein the two or more distance steps may be three or more distance steps. For example, the radar device may be configured to detect the three or more detected radar signals, wherein each detected radar signal of the three or more detected radar signals is assigned to precisely one distance step of the two or more distance steps. For example, each of the three or more distance steps may be assigned to a body region of the plurality of body regions of the human being so that the detected radar signal, assigned to this distance step, of the three or more of the detected radar signals is assigned to said body region. In this case, e.g., the evaluation unit may be configured to determine information about said one of the one or more vital parameters depending on which detected radar signal of the three or more detected radar signals is assigned to which body region of the plurality of body regions.

According to an embodiment, e.g., each of the one or more vital parameters may have assigned thereto one or more of the plurality of body regions. For example, the evaluation unit may be configured to determine information about one of the one or more vital parameters depending on which of the plurality of body regions is assigned to said one of the one or more vital parameters.

In an embodiment, e.g., the evaluation unit may be configured to determine information about the one of the one or more vital parameters depending on which of the two or more reflected radar signals is/are assigned to a body region of the plurality of body regions assigned to said vital parameter of the one or more vital parameters.

According to an embodiment, e.g., the radar device may be configured to select a distance channel from two or more distance channels, which is assigned to one of the two or more distance steps, which is assigned to a body region of the plurality of body regions, which is assigned to said vital parameter so as to detect the one or more detected radar signals.

In an embodiment, e.g., the radar device may be configured to select a second distance channel from two or more distance channels, which is assigned to one of the two or more distance steps, which is assigned to a body region of the plurality of body regions, which is assigned to said vital parameter so as to detect a second of the one or more detected radar signals if a selection of a first distance channel from the two or more distance channels has led to a detection of a first of the one or more detected radar signals being categorized as insufficiently informative by the evaluation unit.

According to an embodiment, e.g., the radar device may be configured to select two or more distance channels, wherein each of the two or more distance channels is assigned to one of two or more distance steps, respectively, each being assigned to a body region, assigned to said vital parameter, of the plurality of body regions so as to detect the two or more detected radar signals.

According to an embodiment, e.g., a vital parameter of the one or more vital parameters may have assigned thereto one or more frequencies and/or one or more frequency ranges. In this case, e.g., the evaluation unit may be configured to determine the information about said vital parameter depending on a periodic change in one of the one or more detected radar signals, comprising a frequency corresponding to one of the one or more frequencies assigned to said vital parameters, and/or being in one of the one or more frequency ranges assigned to said vital parameters.

In an embodiment, e.g., the evaluation unit may be configured to determine the information about one of the one or more vital parameters depending on which body regions of the plurality of body regions have assigned thereto the one or more reflected radar signals in which the periodic change occurs.

According to an embodiment, e.g., the radar device may be realized in a frequency-modulated continuous radar.

In an embodiment, e.g., the system may include one or more further radar devices for emitting further first radar waves and for detecting further reflected radar waves caused by reflection of the further first radar waves at the human being or at the body cover of the human being. In this case, e.g., the evaluation unit may be configured to determine the information about the one or more vital parameters of the human being depending on the further reflected radar waves. In this case, e.g., the one or more further radar devices may be configured to emit the further first radar waves from a different angle relative to the position of the human being.

According to an embodiment, e.g., the radar device may be positioned such that the radar device is closest to a first body region from a plurality of body regions of the human being. In this case, e.g., at least one of the one or more further radar devices may be positioned such that said at least one of the one or more further radar devices is closest to a second body region, different from the first body region, from a plurality of body regions of the human being.

Furthermore, a method for determining information about one or more vital parameters of a human being embodiment is provided according to an. The method includes:
Emitting first radar waves by a radar device.
Determining, by an evaluation unit, the information of the one or more vital parameters of the human being depending on the reflected radar wave. And:
Evaluating, by an evaluation unit, the reflected radar waves to determine the information about the one or more vital parameters of the human being.

The radar device is arranged, relative to the position of the human being, laterally with respect to the human being such that there is a virtual point in a foot of the human being and such that there is a virtual point in the radar device so that the virtual point in the radar device is closer to the virtual point in the foot of the human being than to any virtual point in a navel of the human being. Or the radar device is arranged, relative to the position of the human being, laterally with respect the human being such that there is a virtual point in the radar device so that the virtual point in the radar device is closer to the virtual point in the head of the human being than to any virtual point in the navel of the human being.

According to an embodiment, e.g., the radar device may be arranged relative to the position of the human being such that, instead of behind the human being, the radar device is positioned in front of the human being.

In an embodiment, e.g., the radar device may be arranged, relative to the position of the human being, laterally with respect to the human being such that the radar device is arranged below the navel of the human being and below the foot of the human being, or such that the radar device is arranged above the navel of the human being and above the head of the human being.

According to an embodiment, e.g., the radar may detect one or more detected radar signals depending on the reflected radar waves, wherein each detected radar signal of the one or more detected radar signals is assigned to precisely one distance step of two or more distance steps. For example, each of the two or more distance steps may be assigned to body region of a plurality of body regions of the human being so that the detected radar signal, assigned to this distance step, of the one or more detected radar signals is assigned to said body region. In this case the evaluation unit may be configured to determine information about one of the one or more vital parameters, e.g., depending on which detected radar signal of the one or more detected radar signals is assigned to which body region of the plurality of body regions.

In an embodiment, e.g., each of the two or more distance steps may be assigned to a body region of a plurality of body regions of the human being so that a body of the human being is fully subdivided into the plurality of body regions by the two or more distance steps so that the plurality of body regions together cover the body of the human being.

According to an embodiment, the one or more detected radar signals may be two or more detected radar signals, for example. In this case, the radar device may detect the two or more detected radar signals, for example, wherein each detected radar signals of the two or more detected radar signals is assigned to precisely one distance step of the two or more distance steps. In this case, e.g., each of the two or more distance steps may be assigned to a body region of the plurality of body regions of the human being so that the detected radar signal, assigned to this distance step, of the two or more detected radar signals is assigned to said body region. In this case, e.g., the evaluation unit may determine information about said one of the one or more vital parameters depending on which detected radar signal of the two or more detected radar signals is assigned to which body part of the plurality of body parts.

In an embodiment, e.g., depending on the two or more detected radar signals, the evaluation unit may create a body model of the human being, indicating which radar signal is assigned to which body region of the plurality of body regions of the human being.

In an embodiment, each of the two or more detected radar signals may be assigned to precisely one body region, for example, having assigned no other of the two or one more detected radar signals, of the two or more body regions.

According to an embodiment, e.g., the one or more vital parameters may be two or more vital parameters. In this case, e.g., the evaluation unit may determine information about the two or more vital parameters depending on which detected radar signal of the two or more detected radar signals is assigned to which body region of the plurality of body regions.

In an embodiment, e.g., the two or more detected radar signals may be three or more detected radar signals, wherein the two or more distance steps may be three or more distance steps. For example, the radar device may detect the three or more detected radar signals, wherein each detected radar signal of the three or more detected radar signals is assigned to precisely one distance step of the two or more distance steps. In this case, e.g., each of the three or more distance steps may be assigned to a body region of the plurality of body regions of the human being so that the detected radar signal, assigned to this distance step, of the three or more of the detected radar signals is assigned to said body region. In this case, e.g., the evaluation unit may determine information about said one of the one or more vital parameters depending on which detected radar signal of the three or more detected radar signals is assigned to which body region of the plurality of body regions.

According to an embodiment, each of the one or more vital parameters may have assigned thereto one or more of the plurality of body regions, for example. In this case, e.g., the evaluation unit may determine information about one of the one or more vital parameters depending on which of the plurality of body regions is assigned to said one of the one or more vital parameters.

In an embodiment, e.g., the evaluation unit may determine information about the one of the one or more vital parameters depending on which of the two or more reflected radar signals is/are assigned to a body region of the plurality of body regions assigned to said vital parameter of the one or more vital parameters.

According to an embodiment, e.g., the radar device may select a distance channel from two or more distance channels, which is assigned to one of the two or more distance steps, which is assigned to a body region of the plurality of body regions, which is assigned to said vital parameter so as to detect the one or more detected radar signals.

In an embodiment, e.g., the radar device may select a second distance channel from two or more distance channels, which is assigned to one of the two or more distance steps, which is assigned to a body region of the plurality of body regions, which is assigned to said vital parameter so as to detect a second of the one or more detected radar signals if a selection of a first distance channel from the two or more distance channels has led to a detection of a first of the one or more detected radar signals being categorized as insufficiently informative by the evaluation unit.

According to an embodiment, e.g., the radar device may select two or more distance channels, wherein each of the two or more distance channels is assigned to one of two or more distance steps, respectively, each being assigned to a body region, assigned to said vital parameter, of the plurality of body regions so as to detect the two or more detected radar signals.

According to an embodiment, e.g., a vital parameter of the one or more vital parameters may have assigned thereto one or more frequencies and/or one or more frequency ranges. In this case, the evaluation unit may be configured to determine the information about said vital parameter depending on a periodic change in one of the one or more detected radar signals, for example, comprising a frequency corresponding to one of the one or more frequencies assigned to said vital parameters, and/or being in one of the one or more frequency ranges assigned to said vital parameters.

In an embodiment, e.g., the evaluation unit may be configured to determine the information about one of the one or more vital parameters depending on which body regions of the plurality of body regions have assigned thereto the one or more reflected radar signals in which the periodic change occurs.

According to an embodiment, e.g., the radar device may be realized in a frequency-modulated continuous radar.

In an embodiment, e.g., the system may include one or more further radar devices emitting further first radar waves and detecting further reflected radar waves caused by reflection of the further first radar waves at the human being or at the body cover of the human being. In this case, e.g., the evaluation unit may determine the information about the one or more vital parameters of the human being depending on the further reflected radar waves. In this case, e.g., the one or more further radar devices may emit the further first radar waves from a different angle relative to the position of the human being.

According to an embodiment, e.g., the radar device may be positioned such that the radar device is closest to a first body region from a plurality of body regions of the human being. In this case, e.g., at least one of the one or more further radar devices may be positioned such that said at least one of the one or more further radar devices is closest to a second body region, different from the first body region, from a plurality of body regions of the human being.

Furthermore, at a computer program with a program code for performing one of the above-described methods is provided according to an embodiment.

### BRIEF DESCRIPTION OF THE DRAWINGS

Embodiments of the present invention will be detailed subsequently referring to the appended drawings, in which:

FIG. 1 shows a system for determining information about one of more vital parameters of a human being, according to an embodiment.

FIG. 2 shows a special lateral positioning of the radar device with respect to the human being, according to an embodiment.

FIG. 3 shows a special lateral positioning of the radar device with respect to the human being, according to a second embodiment.

FIG. 4 shows a schematic illustration for a lateral radar view on a prone person, according to an embodiment.

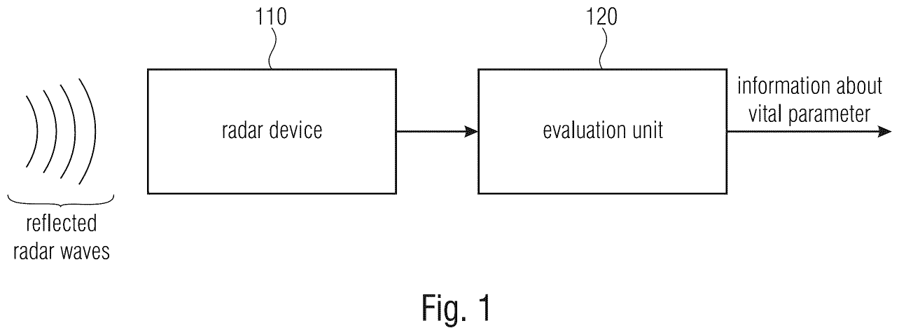

FIG. 1 shows a system for determining information about one or more vital parameters of the human according to an embodiment.

### DETAILED DESCRIPTION OF THE INVENTION

The system includes a radar device 110 for emitting first radar waves and for detecting reflected radar waves caused by a reflection of the first radar waves at the human being or at a body cover of the human being.

In addition, the system includes an evaluation unit 120 for determining information about the one or more vital parameters of the human being depending on the reflected radar wave.

According to the invention, a special positioning of the radar device 110 with respect to the human being provided:

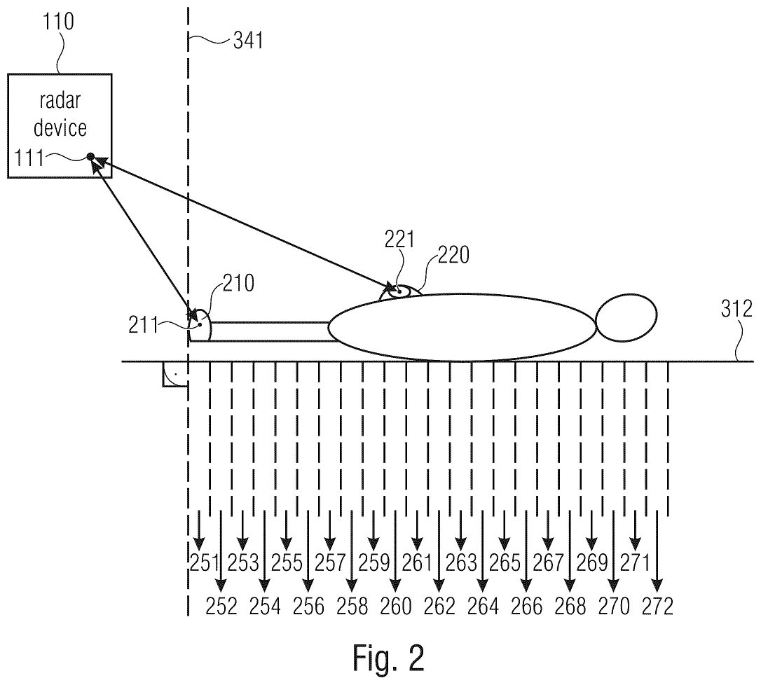

FIG. 2 shows a special lateral positioning of the radar device 110 with respect to the human being, according to a first embodiment. According to the same, the radar device 110 is arranged, relative to a position of the human being, laterally with respect to the human being such that there is a virtual point 211 in a foot 210 of the human being and such that there is a virtual point 111 in the radar device 110 so that the virtual point 111 in the radar device 110 is closer to the virtual point 211 in the foot 210 of the human being than to any virtual point 221 in a navel 220 of the human being.

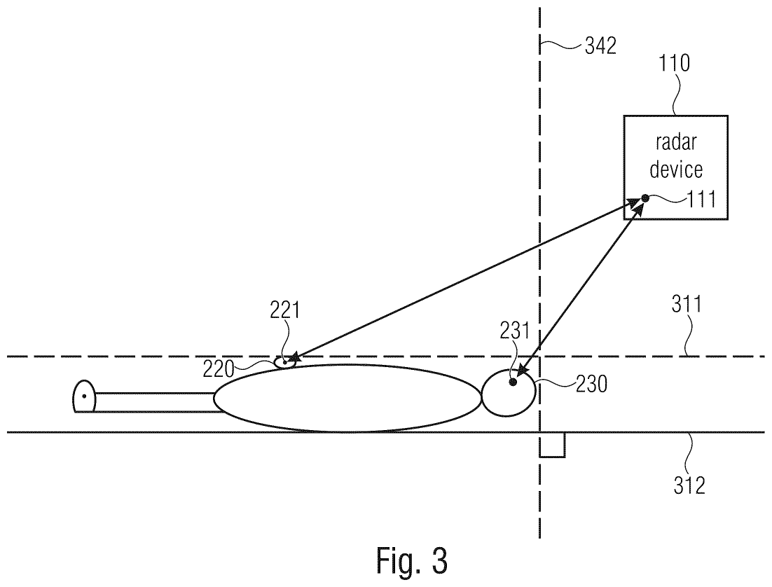

FIG. 3 shows a special lateral positioning of the radar device 110 with respect to the human being, according to a second embodiment. According to the same, the radar device 110 is arranged, relative to the position of the human being, laterally to the human being such that there is a virtual point 231 in a head 230 of the human being and such that there is a virtual point 111 in the radar device 110 so that the virtual point 111 in the radar device 110 is closer to the virtual point 231 in the head 230 of the human being than to any virtual point 221 in the navel 220 of the human being.

Thus, the embodiments of FIG. 2 to FIG. 3 represent a paradigm shift. In general, there is the assumption that the best radar results are achieved if radar waves meet a body, here the body of the human being, perpendicularly, since a maximum approach and distance relative to the radar can be observed from there. In case of this perspective, the direct distance between the radar and the abdomen changes to a maximum during breathing, and this also creates a maximum change of the signal strength of the electromagnetic wave. Embodiments of the invention are now based on the finding that this creates ambiguities and superimpositions that are hard to solve. Thus, the above-defined lateral positioning is chosen.

A further advantage of the lateral positioning may be that an object that is closer to the radar reflects a stronger signal than objects that are further spaced apart. This effect can be compared to a hand that is located directly in front of a light bulb being brighter than a hand that is far away from the light bulb. For example, if the radar is located at the foot end, the feet are closer to the radar and therefore reflect a stronger signal. This helps to determine the small pulse movement at the foot surface very well. The lateral position therefore leads to a “magnification effect”, so to speak, at the body part closest to the radar. This further simplifies pulse detection, which is otherwise more difficult.

According to an embodiment, e.g., the radar device 110 may be arranged relative to the position of the human being such that the radar device 110 is positioned in front of the human being instead of behind the human being. For example, e.g., this is illustrated in FIG. 3 for a prone human being. Since the radar device 110 in FIG. 3 is located above the dotted line 311 and not below the solid line 312, the position of the radar device 110 with respect to the human being in FIG. 3 satisfies this definition. This embodiment is based on the finding of the inventors that vital parameters can often be detected better on movements on the front side of the human being (movement of the chest, abdomen, etc.) than on the backside.

In an embodiment, e.g., the radar device 110 may be arranged, relative to the position of the human being, laterally with respect to the human being such that the radar device 110 is arranged below the navel 220 of the human being and above the foot 210 of the human being. This embodiment is shown in FIG. 2. Since the radar device 110 in FIG. 2 is located left of the dotted line 341 and therefore below the foot 210 (and the navel 220) of the human being, the definition is satisfied. Superimpositions and ambiguities are avoided by a corresponding positioning of the radar device 110 relative to the human being.

Or, in a further embodiment, e.g., the radar device 110 may be arranged, relative to the position of the human being, laterally with respect to the human being such that the radar device 110 is arranged above the navel of the human being and above the head of the human being. This embodiment is shown in FIG. 3. Since the radar device 110 in FIG. 3 is located right of the dotted line 342 and therefore above the head 230 (and the navel 220) of the human being, the definition is satisfied. Superimpositions and ambiguities are avoided by a corresponding positioning of the radar device 110 relative to the human being.

In FIG. 2 and FIG. 3, the dotted lines 341 and 342 may be perpendicular/at right angles to the solid line 312. For example, the solid line 312 may be a lying surface on which the human being is lying. Alternatively, the solid line 312 may also be a virtual line that is parallel to a central line through an imaginary cross-section of the human being.

In FIG. 3, e.g., lines 311 and 312 may be selected such that each of the two lines represents a cross-section through one of two surfaces, respectively, wherein both surfaces are parallel with respect to each other such that the body of the human being is adjacent to both parallel surfaces, such that the body of the human being does not exceed in an upwards direction the surface whose cross-section is represented by line 311, and such that the body of the human being does not, in a downward direction, exceed the surface whose cross-section is represented by line 312.

According to an embodiment, e.g., the radar device 110 may be configured to detect one or more detected radar signals depending on the reflected radar waves, wherein each detected radar signal of the one or more detected radar signals is sent to precisely one distance step of two or more distance steps. In this case, e.g., each of the two or more distance steps may be assigned to a body region of a plurality of body regions of the human being so that the detected radar signal, assigned to this distance step, of the one or more detected radar signals is assigned to said body region. In this case, e.g., the evaluation unit 120 may be configured to determine information about the one or more vital parameters depending on which detected radar signal of the one or more detected radar signals is assigned to which body region of the plurality of body regions. By means of this embodiment, one or more detected radar signals assigned to a special body region of a multitude of body regions may be captured (cf. FIG. 2; body regions 251, 252, 253, 254, 255, 256, 257, 258, 259, 260, 261, 262, 263, 264, 265, 266, 267, 268, 269, 270, 271, 272).

In an embodiment, each of the two or more distance steps may be assigned to a body region, e.g., of plurality of body regions of the human being so that a body of the human being is fully subdivided into a plurality of body regions by the two or more distance steps so that the plurality of body regions together cover the body of the human being. For this, see FIG. 2 and the body regions 251, 252, 253, 254, 255, 256, 257, 258, 259, 260, 261, 262, 263, 264, 265, 266, 267, 268, 269, 270, 271, 272. Thus, this embodiment (indirectly) creates an unambiguous mapping of a detected radar signal to one of the body regions 251, 252, 253, 254, 255, 256, 257, 258, 259, 260, 261, 262, 263, 264, 265, 266, 267, 268, 269, 270, 271, 272.

According to an embodiment, e.g., the one or more detected radar signals may be two or more detected radar signals. In this case, e.g., the radar device 110 may be configured to detect the two or more detected radar signals, wherein each detected radar signal of the two or more detected radar signals is assigned to precisely one distance step of the two or more distance steps. Each of the two or more distance steps may be assigned to a body region of the plurality of body regions of the human being, e.g., so that the detected radar signal, assigned to this distance step, of the two or more detected radar signals is assigned to said body region. In this case, e.g., the evaluation unit 120 may be configured to determine information about said one of the one or more vital parameters depending on which detected radar signal of the two or more detected radar signals is assigned to which body region of the plurality of body regions. This embodiment makes it possible to obtain radar information for a vital parameter from different body regions, and/or to simultaneously detect radar information for two or more vital parameters.

In an embodiment, e.g., depending on the two or more detected radar signals, the evaluation unit 120 may be configured to create a body model of the human being, indicating which radar signal is assigned to which body region of the plurality of body regions. For example, a typical breathing frequency may be captured mainly in the lung and abdominal regions, but rather not at the arms and feet of the human being. However, it should be possible to determine movements caused by a pulse at the whole body. By means of a corresponding analysis of the two or more detected radar signals, typical movement frequencies allow determining where the abdominal and lung regions of a human being is located and where the limbs of a human being are located. Accordingly, a body model may be created by evaluating typical movement frequencies.

In an embodiment, e.g., each of the two or more detected radar signals may be assigned to precisely one body region, having assigned no other of the two or more detected radar signals, of the two or more body regions. This embodiment enables an unambiguous mapping of radar signals to body regions.

According to an embodiment, e.g., the one or more vital parameters may be two or more vital parameters. In this case, e.g., the evaluating unit 120 may be configured to determine information about the two or more vital parameters depending on which detected radar signal of the two or more detected radar signals is assigned to which body region of the plurality of body regions. Thus, in such an embodiment, information may be obtained simultaneously with respect to more than one vital parameter.

In an embodiment, the two or more detected radar signals may be three or more detected radar signals, for example, wherein the two or more distance steps are three or more distance steps. For example, the radar device 110 may be configured to detect the three or more detected radar signals, wherein each detected radar signal of the three or more detected radar signals is assigned to precisely one distance step of the three or more distance steps. For example, each of the three or more distance steps may be assigned to a body region of the plurality of body regions of the human being so that the detected radar signal, assigned to this distance step, of the three or more detected radar signals is assigned to said body region. For example, the evaluation unit 120 may be configured to determine information about said one of the one or more vital parameters depending on which detected radar signal of the three or more detected radar signals is assigned to which body region to the plurality of body regions.

According to an embodiment, e.g., each of the one or more vital parameters may have assigned thereto two or more body regions of the plurality of body regions. For example, the evaluation unit 120 may be configured to determine information about one of the one or more vital parameters depending on which of the plurality of body regions is assigned to said one of the one or more vital parameters.

In an embodiment, e.g., the evaluation unit 120 may be configured to determine information about one of the one or more vital parameters depending on which of the two or more reflected radar signals is/are assigned to a body region of the plurality of body regions assigned to said vital parameter of the one or more vital parameters.

According to an embodiment, e.g., the radar device 110 may be configured to select a distance channel from two or more distance channels, which is assigned to one of the two or more distance steps, which is assigned to a body region of the plurality of body regions, which is assigned to said vital parameter so as to detect one of the one or more detected radar signals.

In an embodiment, e.g., the radar device 110 may be configured to select a second distance channel from two or more distance channels, which is assigned to one of the two or more distance steps, which is assigned to a body region of the plurality of body regions, which is assigned to said vital parameter, so as to detect a second of the one or more detected radar signals if a selection of a first distance channel from the two or more distance channels has led to the detection of a first of the one or more detected radar signals being categorized as insufficiently informative by the evaluation unit 120.

According to an embodiment, e.g., the radar device 110 may be configured to select two or more distance channels, wherein each of the two or more distance channels is assigned to one of two or more distance steps, respectively, each being assigned to a body region, assigned to said vital parameter, of the plurality of body regions so as to detect the two or more detected radar signals.

According to an embodiment, e.g., a vital parameter of the one or more vital parameters may have assigned thereto one or more frequencies and/or one or more frequency ranges. In this case, e.g., the evaluation unit 120 may be configured to determine the information about said vital parameter depending on a periodic change in one of the one or more detected radar signals, comprising a frequency corresponding to one of the one or more frequencies assigned to said vital parameters, and/or being in one of the one or more frequency ranges assigned to said vital parameters.

In an embodiment, e.g., the evaluation unit 120 may be configured to determine the information about one of the one or more vital parameters depending on which body regions of the plurality of body regions have assigned thereto the one or more reflected radar signals in which the periodic change occurs.

According to an embodiment, the radar device 110 may be realized as a frequency-modulated continuous wave radar.

In an embodiment, e.g., the system may include one or more further radar devices for emitting further first radar waves and for detecting further reflected radar waves caused by reflection of the further first radar waves at the human being or at the body cover of the human being. In this case, e.g., the evaluation unit 120 may be configured to determine the information about the one or more vital parameters of the human being depending on the further reflected radar waves. In this case, e.g., the one or more further radar devices may be configured to emit the further first radar waves from a different angle relative to the position of the human being.

According to an embodiment, the radar device 110 may be positioned such that the radar device 110 is closest to a first body region (body portion) from a plurality of body regions (body portions) of the human being. In this case, e.g., at least one of the one or more further radar devices may be positioned such that said at least one of the one or more further radar devices is/are closest to a second body region (body portion), different from the first body region, from a plurality of body regions of the human being.

Such an embodiment also particularly uses the above-described effect that an object closer to the radar reflects a stronger signal than objects farther away. If one or more further radar signals emitting from a different angle relative to the position of the human being are used, the proximity of the various body regions to the one or more further radar devices also changes in comparison to the first radar device. As a result, the one or more additional radar devices may be used to select a different measurement accuracy for each of the body regions by differently setting the distance between the body region and the respective radar device. In this way, each of the radar devices may be focused on the specific body region.

Special embodiments of the invention are subsequently described in detail.

Embodiments provide a fixed position of a radar, having a lateral view onto the human being from this position. This enables a subdivision of the human being into individual distinguishable portions, e.g. from feet to head. These distinguishable portions are the basis for separating heartbeat and breathing.

In principle, a radar measures distances by dividing the space in front of the radar into individual distance steps.

For example, the width of the distance steps may depend on the distance resolution of the radar. If an object is located in such a portion, the associated measuring values have a high value.

In an embodiment, e.g., the distance steps may be 5 cm.

In another embodiment, e.g., an upper-body-lower-body model may be used, e.g., wherein the distance steps could rather be 25 cm instead of 5 cm. However, compared to the distance steps of 5 cm, the separation of the vital parameters becomes more difficult in case of distance steps of 25 cm.

If the radar observes the person laterally (e.g. a person lying on a bed is observed from a foot end of the bed), the entire body of the human being is subdivided into individual portions. A coarse model of the body can be created on this basis. If the person breathes, the values close to the abdominal region change periodically. If the heart beats, the pulse may be detected, e.g. at the upper chest, on the basis of a different frequency in the breathing and a lower amplitude. Due to a spatial segmentation of the radar response, the pulse may also be captured at other body regions, e.g. at the foot. The basis of the vital parameters is located in different distance steps and is therefore already spatially separated.

Due to this spatial segmentation, the desired signal may be captured at different body positions in connection with an analysis and evaluation unit 120. This unit may have different implementations.

A first embodiment may provide a direct selection of a distance channel that best represents the desired body signal. The direct selection of the distance channel therefore determines a signal that is particularly free of interference from other body signals.

A second embodiment uses several distance channels from which the desired body signal is computed by means of multi-channel processing, e.g. by means of correlation.

A third embodiment uses a method like the first embodiment or the second embodiment, where a body model that uses knowledge about the position of the body and/or knowledge about the desired signal and/or about possible interferences at specific body positions is used as a basis.

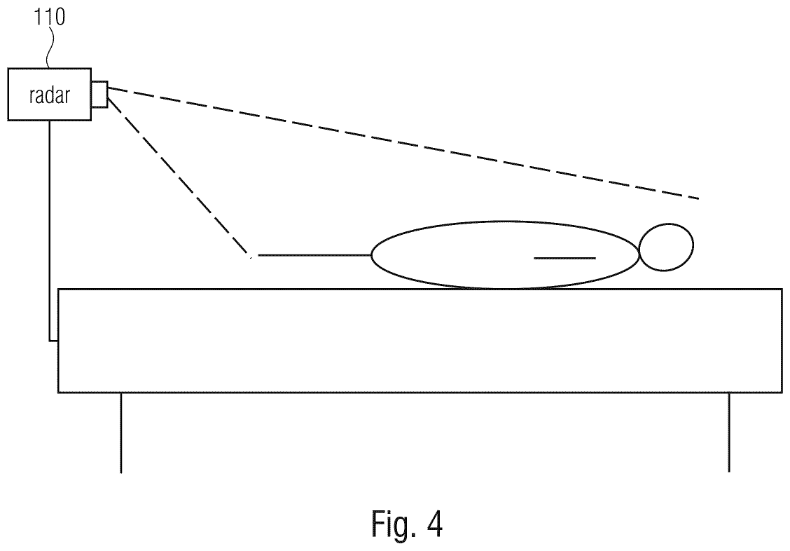

FIG. 4 shows a schematic illustration for a lateral radar view onto a prone person according to an embodiment.

In this case, an embodiment on the basis of FIG. 4 would be the measurement from the foot end of the bed. In this case, the radar observes a prone human being whose feet are closest to the radar and whose head is furthest away from the radar.

For example, the radar subdivides the person in five-centimeter steps, starting from the feet to the head, for example.

For example, a coarse 3D model of the person may be created by means of several radar antenna in the device.

For example, then the person breathes, a periodic movement in the frequency range of human breathing should be observable approximately in the center of the body. For example, in the case of a heartbeat, the pulse should be visible across the entire body; thus, e.g., across all distance steps that could have been assigned to the human being, since the entire body has a pulse.

For example, if the human being moves his/her foot while sleeping (“restless leg syndrome”), movement should be detected close to the foot end of the bed.

For example, these movements may already be subdivided into individual distance steps during the measuring process. As a result, a separation of all these movements is less complex in the evaluation than when capturing the data above or below the body, wherein the data is superimposed more strongly. Better separation of the signals during capturing enables robust detection of the parameters.

In an embodiment, a radar may be located in a lateral position with respect to a prone human being, e.g. at an edge of the bed, so that a prone human being can be observed laterally from the position of the radar. In this case, the human being may be subdivided into individual distance steps. In this case, e.g., a 3D model for the extraction of vital parameters may be created from the radar data.

Advantageously, the position of a radar transceiver (or a radar transmitter and a radar receiver) may be located above the head or at the foot end of the bed.

Some embodiments may distinguish in a dedicated way between abdominal breathing and chest breathing in the result, for example.

The effect of the lateral view and the subdivision of the human being (in particular in several distance steps) simplifies the evaluation of vital parameters by not having a superimposed complex signal in the raw data. This renders a separation of this signal into individual vital parameters by means of complex algorithms unnecessary, and the evaluation becomes more robust overall.

Breaks in the detection of vital parameters are still possible; however, they are less probable than in case of a more complex underlying signal. In addition, breaks may be determined and interpreted at a model of the body. For example, in the model, the heartbeat should reach each part of the body, while breathing movements should only be located at the center of the body. The validity and comprehensibility of the individual results are higher in this approach than in previous approaches.

Further embodiments of the invention are described in the following:

In embodiments, a human being can be divided (subdivided) using radar into different distance steps that can be used to detect vital parameters along the body.

According to embodiments, e.g., this can be made possible by a lateral view (from the radar) of the human being, advantageously from a head-to-feet or feet-to-head perspective. For example, the radar observes the person at an angle.

Embodiments use distance bins, which are several distance channels in which vital parameters are visible. Vital parameters in the distance bins may be detected, e.g., on the basis of changes in the values in a frequency range that is normal for human beings over time. For example, breathing may be in a range of 0.1 Hz-0.58 Hz, and heartbeats may be in a range of 0.8 Hz-2 Hz.

According to embodiments, recognition of a human body may be done on the basis of the vital parameters in the spatially segmented radar signals.

For example, each part of the human body should have a pulse and reflect the radar radiation. Radar radiation penetrates fabric, e.g. clothing, bed covers, which is why the body surface remains visible. The pulse may be used to estimate the boundaries of the body, in addition to normal object recognition by the radar (reflection of objects). The upper body, where the pulse is masked by breathing, may be detected by breathing.

Embodiments may use the fact that the movement of the body is visible on the basis of the movement of the distance bins with recognized vital parameters.

According to embodiments, a simplified evaluation can be carried out by observing the vital parameters at different locations along the body, e.g. a pulse at the foot and/or forehead, and/or a breathing along the upper body.

In embodiments, a simpler extraction of the vital parameters may be realized by a smaller superimposition of the movements at certain body parts. For example, breathing may be clearly extracted at the chest, a heartbeat may be clearly extracted at the forehead/feet.

According to embodiments, a more robust vital parameter detection may be realized by multiple vital parameter locations.

For example, multiple vital parameter locations may be compared with each other. For example, the same pulse rate should be present along the body for a human being.

For example, in embodiments, alternative locations may be selected if the usual vital parameters locations are obscured.

In embodiments, an additional extension of the body model may be provided by observing the human being from multiple angles, e.g. by using two (or more) radars and/or by using multiple antennas. In this way, a human being may be simultaneously divided/subdivided into distance steps from different angles.

According to embodiments, e.g., the creation of a 3D model may be realized on the basis of this information.

In embodiments, better data quality (e.g. a better signal-to-noise ratio, SNR) may be realized by observing the same situation from different angles and, e.g., comparing the measurements.

According to embodiments, e.g., more information may be obtained about an ongoing movement of a body, for example of arms and/or legs, since the movement is observed from different directions.

In embodiments, e.g., a specified radar position may be used for additional information about the situation. For example, the radar may be deliberately positioned at the foot end, which means that the foot should be seen first in the evaluation. This may be taken into account in the evaluation.

According to embodiments, e.g., a model of the body can be created and/or used to interpret the information from the spatially segmented radar signals. For example, this information may be used to recognize position, expected movements, and pathological conditions on the basis of the body model.

In embodiments, e.g., recognition of vital parameters may be done not only by frequency, but also by a probable location of the vital parameter relative to the rest of the body model. For example, a normal body model, in which breathing also occurs at the foot, may be categorized as inadmissible.

According to embodiments, recognition of the upper body may be done recognition of breathing and heartbeat signals in closely spaced distance bins, for example.

In embodiments, e.g., information about the supply of the body may be obtained based on the presence of the pulse along the body. For example, it may be determined that there is no pulse at the feet, but that there is a pulse everywhere else.

For example, embodiments of a model of a body may be based on an assumption that the human being cannot change indefinitely rapidly. For example, this may further assume that the location and frequency of vital parameters cannot fluctuate arbitrarily between measurements.

According to embodiments, e.g., information serving to differentiate between obstructive and central apnea may be obtained by observing breathing along the upper body. For example, in central apnea, there may be no abdominal movement; in obstructive apnea, for example, abdominal and thoracic breathing may not be synchronized.

For example, a special embodiment may use a FMCW (frequency-modulated continuous wave) radar, e.g. in the frequency range of 60-64 GHz. In this way, e.g., information used to recognize sleep, respiratory, movement, and pulse disorders may be obtained.

According to embodiments, e.g., more information about breathing events, such as coughing/snoring, may be obtained by observing the entire body, e.g. snoring at the larynx and abdomen.

For example, areas of application for the contactless measurement of vital parameters by observing and subdividing a sleeping human being from the side are sleep measurements. Here, the human being or human beings are lying in a bed, and the radar is attached to the edge of the bed.

Specific situations for this setup could include clinical sleep monitoring to obtain intermediate results that could be used for subsequent diagnosis (e.g. sleep disorders, circulatory disorders, movement disorders, mental illnesses).

For example, another field of application could be home or out-patient sleep monitoring to obtain intermediate results that could be used for subsequent diagnosis or to obtain results that could be used for private purposes.

another field of application could be clinical or home monitoring, such as monitoring the effectiveness of positive air pressure systems, e.g. for the treatment of apnea.

For example, another field of application could be sleep studies in science, e.g., to gain insights into the correlation between various vital parameters.

For example, another field of application could be monitoring of people, e.g. during a prison sentence, e.g. in a prison.

For example, a further field of application could be contactless capturing and differentiation of obstructive and central breathing pauses (apnea) and hypopnea.

For example, another field of application could be a combination with other sensor technology (e.g. audio, e.g. EEG) as an alternative to polysomnography.

Even though some aspects have been described within the context of a device, it is understood that said aspects also represent a description of the corresponding method, so that a block or a structural component of a device is also to be understood as a corresponding method step or as a feature of a method step. By analogy therewith, aspects that have been described within the context of or as a method step also represent a description of a corresponding block or detail or feature of a corresponding device. Some or all of the method steps may be performed while using a hardware device, such as a microprocessor, a programmable computer or an electronic circuit. In some embodiments, some or several of the most important method steps may be performed by such a device.

Depending on specific implementation requirements, embodiments of the invention may be implemented in hardware or in software. Implementation may be effected while using a digital storage medium, for example a floppy disc, a DVD, a Blu-ray disc, a CD, a ROM, a PROM, an EPROM, an EEPROM or a FLASH memory, a hard disc or any other magnetic or optical memory which has electronically readable control signals stored thereon which may cooperate, or cooperate, with a programmable computer system such that the respective method is performed. This is why the digital storage medium may be computer-readable.

Some embodiments in accordance with the invention thus comprise a data carrier which comprises electronically readable control signals that are capable of cooperating with a programmable computer system such that any of the methods described herein is performed.

Generally, embodiments of the present invention may be implemented as a computer program product having a program code, the program code being effective to perform any of the methods when the computer program product runs on a computer.

The program code may also be stored on a machine-readable carrier, for example.

Other embodiments include the computer program for performing any of the methods described herein, said computer program being stored on a machine-readable carrier. In other words, an embodiment of the inventive method thus is a computer program which has a program code for performing any of the methods described herein, when the computer program runs on a computer.

A further embodiment of the inventive methods thus is a data carrier (or a digital storage medium or a computer-readable medium) on which the computer program for performing any of the methods described herein is recorded. The data carrier, the digital storage medium, or the recorded medium are typically tangible, or non-volatile.

A further embodiment of the inventive method thus is a data stream or a sequence of signals representing the computer program for performing any of the methods described herein. The data stream or the sequence of signals may be configured, for example, to be transferred via a data communication link, for example via the internet.

A further embodiment includes a processing means, for example a computer or a programmable logic device, configured or adapted to perform any of the methods described herein.

A further embodiment includes a computer on which the computer program for performing any of the methods described herein is installed.

A further embodiment in accordance with the invention includes a device or a system configured to transmit a computer program for performing at least one of the methods described herein to a receiver. The transmission may be electronic or optical, for example. The receiver may be a computer, a mobile device, a memory device or a similar device, for example. The device or the system may include a file server for transmitting the computer program to the receiver, for example.

In some embodiments, a programmable logic device (for example a field-programmable gate array, an FPGA) may be used for performing some or all of the functionalities of the methods described herein. In some embodiments, a field-programmable gate array may cooperate with a microprocessor to perform any of the methods described herein. Generally, the methods are performed, in some embodiments, by any hardware device. Said hardware device may be any universally applicable hardware such as a computer processor (CPU), or may be a hardware specific to the method, such as an ASIC.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1. A system for determining information about one or more vital parameters of a human being, the system comprising:
a radar device for emitting first radar waves and detecting reflected radar waves caused by reflection of the first radar waves at the human being or at a body cover of the human being, and
an evaluation unit for determining information about the one or more vital parameters of the human being depending on the reflected radar waves,
wherein the radar device is arranged, relative to the position of the human being, laterally with respect to the human being such that there is a virtual point in a foot of the human being and such that there is a virtual point in the radar device so that the virtual point in the radar device is closer to the virtual point in the foot of the human being than to any virtual point in a navel of the human being; or
wherein the radar device is arranged, relative to the position of the human being, laterally with respect the human being such that there is a virtual point in a head of the human being and such that there is a virtual point in the radar device so that the virtual point in the radar device is closer to the virtual point in the head of the human being than to any virtual point in the navel of the human being.

2. The system according to claim 1,
wherein the radar device is arranged relative to the position of the human being such that, instead of behind the human being, the radar device is positioned in front of the human being.

3. The system according to claim 1,
wherein the radar device is arranged, relative to the position of the human being, laterally with respect to the human being such that the radar device is arranged below the navel of the human being and below the foot of the human being, or such that the radar device is arranged above the navel of the human being and above the head of the human being.

4. The system according to claim 1,
wherein the radar device is configured to detect one or more detected radar signals depending on the reflected radar waves, wherein each detected radar signal of the one or more detected radar signals is assigned to precisely one distance step of two or more distance steps,
wherein each of the two or more distance steps is assigned to body region of a plurality of body regions of the human being so that the detected radar signal, assigned to this distance step, of the one or more detected radar signals is assigned to said body region,
wherein the evaluation unit is configured to determine information about one of the one or more vital parameters depending on which detected radar signal of the one or more detected radar signals is assigned to which body region of the plurality of body regions.

5. The system according to claim 4,
wherein each of the two or more distance steps is assigned to a body region of a plurality of body regions of the human being so that a body of the human being is fully subdivided into the plurality of body regions by the two or more distance steps so that the plurality of body regions together cover the body of the human being.

6. The system according to claim 4,
wherein the one or more detected radar signals are two or more detected radar signals,
wherein the radar device is configured to detect the two or more detected radar signals, wherein each detected radar signals of the two or more detected radar signals is assigned to precisely one distance step of the two or more distance steps,
wherein each of the two or more distance steps is assigned to a body region of the plurality of body regions of the human being so that the detected radar signal, assigned to this distance step, of the two or more detected radar signals is assigned to said body region,
wherein the evaluation unit is configured to determine information about said one of the one or more vital parameters depending on which detected radar signal of the two or more detected radar signals is assigned to which body part of the plurality of body parts.

7. The system according to claim 6,
wherein, depending on the two or more detected radar signals, the evaluation unit is configured to create a body model of the human being, indicating which radar signal is assigned to which body region of the plurality of body regions of the human being.

8. The system according to claim 6,
wherein each of the two or more detected radar signals is assigned to precisely one body region, having assigned no other of the two or one more detected radar signals, of the two or more body regions.

9. The system according to claim 6,
wherein the one or more vital parameters are two or more vital parameters,
wherein the evaluation unit is configured to determine information about the two or more vital parameters depending on which detected radar signal of the two or more detected radar signals is assigned to which body region of the plurality of body regions.

10. The system according to claim 6,
wherein the two or more detected radar signals are three or more detected radar signals, wherein the two or more distance steps are three or more distance steps,
wherein the radar device is configured to detect the three or more detected radar signals, wherein each detected radar signal of the three or more detected radar signals is assigned to precisely one distance step of the two or more distance steps,
wherein each of the three or more distance steps is assigned to a body region of the plurality of body regions of the human being so that the detected radar signal, assigned to this distance step, of the three or more of the detected radar signals is assigned to said body region,
wherein the evaluation unit is configured to determine information about said one of the one or more vital parameters depending on which detected radar signal of the three or more detected radar signals is assigned to which body region of the plurality of body regions.

11. The system according claim 4,
wherein each of the one or more vital parameters has assigned thereto one or more of the plurality of body regions,
wherein the evaluation unit is configured to determine information about one of the one or more vital parameters depending on which of the plurality of body regions are assigned to said one of the one or more vital parameters.

12. The system according to claim 4,
wherein each of the one or more vital parameters has assigned thereto one or more of the plurality of body regions,
wherein the evaluation unit is configured to determine information about one of the one or more vital parameters depending on which of the plurality of body regions are assigned to said one of the one or more vital parameters, and
wherein the evaluation unit is configured to determine information about the one of the one or more vital parameters depending on which of the two or more reflected radar signals is/are assigned to a body region of the plurality of body regions assigned to said vital parameter of the one or more vital parameters.

13. The system according to claim 11,
wherein the radar device is configured to select a distance channel from two or more distance channels, which is assigned to one of the two or more distance steps, which is assigned to a body region of the plurality of body regions, which is assigned to said vital parameter so as to detect the one or more detected radar signals.

14. The system according to claim 13,
wherein the radar device is configured to select a second distance channel from two or more distance channels, which is assigned to one of the two or more distance steps, which is assigned to a body region of the plurality of body regions, which is assigned to said vital parameter so as to detect a second of the one or more detected radar signals if a selection of a first distance channel from the two or more distance channels has led to a detection of a first of the one or more detected radar signals being categorized as insufficiently informative by the evaluation unit.

15. The system according to claim 6,
wherein each of the one or more vital parameters has assigned thereto one or more of the plurality of body regions,
wherein the evaluation unit is configured to determine information about one of the one or more vital parameters depending on which of the plurality of body regions are assigned to said one of the one or more vital parameters, and
wherein the radar device is configured to select two or more distance channels, wherein each of the two or more distance channels is assigned to one of two or more distance steps, respectively, each being assigned to a body region, assigned to said vital parameter, of the plurality of body regions so as to detect the two or more detected radar signals.

16. The system according to claim 4,
wherein a vital parameter of the one or more vital parameters has assigned thereto one or more frequencies and/or one or more frequency ranges,
wherein the evaluation unit is configured to determine the information about said vital parameter depending on a periodic change in one of the one or more detected radar signals, comprising a frequency corresponding to one of the one or more frequencies assigned to said vital parameters, and/or being in one of the one or more frequency ranges assigned to said vital parameters.

17. The system according to claim 16,
wherein the evaluation unit is configured to determine the information about one of the one or more vital parameters depending on which body regions of the plurality of body regions have assigned thereto the one or more reflected radar signals in which the periodic change occurs.

18. The system according to claim 1,
wherein the radar device is realized in a frequency-modulated continuous radar.

19. The system according to claim 1,
wherein the system comprises one or more further radar devices for emitting further first radar waves and for detecting further reflected radar waves caused by reflection of the further first radar waves at the human being or at the body cover of the human being,
wherein the evaluation unit is configured to determine the information about the one or more vital parameters of the human being depending on the further reflected radar waves,
wherein the one or more further radar devices is/are configured to emit the further first radar waves from a different angle relative to the position of the human being.

20. The system according to claim 19,
wherein the radar device is positioned such that the radar device is closest to a first body region from a plurality of body regions of the human being, and
wherein at least one of the one or more further radar devices is positioned such that said at least one of the one or more further radar devices is closest to a second body region, different from the first body region, from a plurality of body regions of the human being.

21. A method for determining information about one or more vital parameters of a human being, the method comprising:
emitting first radar waves by a radar device,
detecting, by the radar device, reflected radar waves caused by reflection of the first radar waves at the human being or at a body cover of the human being, and
determining, by an evaluation unit, the information of the one or more vital parameters of the human being depending on the reflected radar wave, and
wherein the radar device is arranged, relative to the position of the human being, laterally with respect to the human being such that there is a virtual point in a foot of the human being and such that there is a virtual point in the radar device so that the virtual point in the radar device is closer to the virtual point in the foot of the human being than to any virtual point in a navel of the human being; or
wherein the radar device is arranged, relative to the position of the human being, laterally with respect the human being such that there is a virtual point in a head of the human being and such that there is a virtual point in the radar device so that the virtual point in the radar device is closer to the virtual point in the head of the human being than to any virtual point in the navel of the human being.

22. The method according to claim 21,
wherein the radar device is arranged, relative to the position of the human being, laterally with respect to the human being such that the radar device is arranged below the navel of the human being and below the foot of the human being, or such that the radar device is arranged above the navel of the human being and above the head of the human being.

23. The method according to claim 21,
wherein the radar device detects one or more detected radar signals depending on the reflected radar waves, wherein each detected radar signal of the one or more detected radar signals is assigned to precisely one distance step of two or more distance steps,
wherein each of the two or more distance steps is assigned to body region of a plurality of body regions of the human being so that the detected radar signal, assigned to this distance step, of the one or more detected radar signals is assigned to said body region,
wherein the evaluation unit is configured to determine information about one of the one or more vital parameters depending on which detected radar signal of the one or more detected radar signals is assigned to which body region of the plurality of body regions.

24. The method according to claim 23,
wherein each of the two or more distance steps is assigned to a body region of a plurality of body regions of the human being so that a body of the human being is fully subdivided into the plurality of body regions by the two or more distance steps so that the plurality of body regions together cover the body of the human being.

25. The method according to claim 23,
wherein the one or more detected radar signals are two or more detected radar signals,
wherein the radar device detects the two or more detected radar signals, wherein each detected radar signals of the two or more detected radar signals is assigned to precisely one distance step of the two or more distance steps,
wherein each of the two or more distance steps is assigned to a body region of the plurality of body regions of the human being so that the detected radar signal, assigned to this distance step, of the two or more detected radar signals is assigned to said body region,
wherein the evaluation unit determines information about said one of the one or more vital parameters depending on which detected radar signal of the two or more detected radar signals is assigned to which body part of the plurality of body parts.

26. The method according to claim 25,
wherein, depending on the two or more detected radar signals, the evaluation unit creates a body model of the human being, indicating which radar signal is assigned to which body region of the plurality of body regions of the human being.

27. The method according to claim 23,
wherein each of the one or more vital parameters has assigned thereto one or more of the plurality of body regions,
wherein the evaluation unit is configured to determine information about one of the one or more vital parameters depending on which of the plurality of body regions are assigned to said one of the one or more vital parameters.

28. The method according to claim 23,
wherein a vital parameter of the one or more vital parameters has assigned thereto one or more frequencies and/or one or more frequency ranges,
wherein the evaluation unit is configured to determine the information about said vital parameter depending on a periodic change in one of the one or more detected radar signals, comprising a frequency corresponding to one of the one or more frequencies assigned to said vital parameters, and/or being in one of the one or more frequency ranges assigned to said vital parameters.

29. The method according to claim 28,
wherein the evaluation unit is configured to determine the information about one of the one or more vital parameters depending on which body regions of the plurality of body regions have assigned thereto the one or more reflected radar signals in which the periodic change occurs.

30. The method according to claim 21,
wherein the system comprises one or more further radar devices emitting further first radar waves and detecting further reflected radar waves caused by reflection of the further first radar waves at the human being or at the body cover of the human being,
wherein the evaluation unit determines the information about the one or more vital parameters of the human being depending on the further reflected radar waves,
wherein the one or more further radar devices emits the further first radar waves from a different angle relative to the position of the human being.

31. A method according to claim 30,
wherein the radar device is positioned such that the radar device is closest to a first body region from a plurality of body regions of the human being,
wherein at least one of the one or more further radar devices is positioned such that said at least one of the one or more further radar devices is closest to a second body region, different from the first body region, from a plurality of body regions of the human being.

32. A non-transitory digital storage medium having a computer program stored thereon to perform the method for determining information about one or more vital parameters of a human being, the method comprising:
emitting first radar waves by a radar device,
detecting, by the radar device, reflected radar waves caused by reflection of the first radar waves at the human being or at a body cover of the human being, and
determining, by an evaluation unit, the information of the one or more vital parameters of the human being depending on the reflected radar wave, and
wherein the radar device is arranged, relative to the position of the human being, laterally with respect to the human being such that there is a virtual point in a foot of the human being and such that there is a virtual point in the radar device so that the virtual point in the radar device is closer to the virtual point in the foot of the human being than to any virtual point in a navel of the human being; or
wherein the radar device is arranged, relative to the position of the human being, laterally with respect the human being such that there is a virtual point in a head of the human being and such that there is a virtual point in the radar device so that the virtual point in the radar device is closer to the virtual point in the head of the human being than to any virtual point in the navel of the human being,
when said computer program is run by a computer.
